(19) United States
(12) Patent Application Publication
(10) Pub. No.: US 2022/0409810 A1
(43) Pub. Date: Dec. 29, 2022

(54) Device For Inserting A Needle

(71) Applicant: Nemera La Verpillière, LA VERPILLIERE (FR)

(72) Inventors: Loïck Cassagne, Saint-Alban-de-Roche (FR); Thibaut Pierquin, CALUIRE ET CUIRE (FR); Pierre Pintus, CRACHIER (FR); Gaëtan Grenot, LYON (FR)

(21) Appl. No.: 17/780,353

(22) PCT Filed: Nov. 26, 2020
(86) PCT No.: PCT/EP2020/084534

§ 371 Date: May 26, 2022

(30) Foreign Application Priority Data
Nov. 26, 2019 (FR) FR1913271

(51) Int. Cl. A61M 5/158; A61M 5/46; A61M 5/142

(52) U.S. Cl. CPC A61M 5/158; A61M 5/46; A61M 5/14244; A61M 2005/1585

(57) ABSTRACT

The insertion device for inserting a needle into a site having a body provided with a bearing wall for resting on the site, a needle support on which a needle is mounted, the needle support being mounted so as to be able to move relative to the bearing wall between a position before insertion, at least one insertion position, and at least one retracted position, an electric motor comprising a rotation shaft, the electric motor being intended to move the needle support in translation between the different positions of the needle support, a detection unit configured to provide a value relative to the operation of the electric motor, and an electronic unit configured to cooperate with the detection unit in order to control the depth of insertion of the needle.

### FIELD OF THE INVENTION

The invention relates to a device for inserting a needle used to dispense a product into the site where the needle was inserted, especially by using a catheter (or cannula). The invention also relates to a method for using such an insertion device.

### BACKGROUND OF THE INVENTION

The state of the art, in particular document W02018152073 already describes a device for inserting a cannula in order to insert a cannula to a preset depth into the skin of a patient. A medicinal product is then dispensed via the cannula which remains partially under the skin. The device as described in this document cannot be used to adjust the position of the cannula under the skin. In other words, the insertion depth cannot be adjusted. However, depending on the morphologies, for example if there is more or less body fat, the medication may be injected at an undesired depth. In addition, it should be possible to use such a device for inserting a cannula for several different types of injection, such as a subcutaneous injection or an intramuscular injection. Thus, being able to adjust the insertion depth would represent a particularly interesting function to adapt the insertion depth according to the type of injection required.

The invention aims in particular to provide a device for inserting a needle that can be used in different ways.

### SUMMARY OF THE INVENTION

This invention therefore relates to an insertion device for inserting a needle into a site, comprising:
a body provided with a bearing wall for bearing on the site,
a needle support on which a needle is mounted, the needle support being mounted so as to be movable with respect to the bearing wall between:
a pre-insertion position in which the needle is retracted with respect to the bearing wall,
at least one insertion position in which the needle protrudes with respect to the bearing wall, and
at least one retracted position in which the needle is once again retracted with respect to the bearing wall,
an electric motor comprising a rotation shaft, the electric motor being configured to move the needle support in translation between the different positions of the needle support,
detection means configured to provide a value relative to the operation of the electric motor,
electronic means configured to cooperate with the detection means in order to control the depth of insertion of the needle.

By using means for detecting at least one parameter of the electric motor, the displacement of the needle support can be controlled and the needle support can be moved to different positions, in particular the insertion position, more precisely.

Thus, several insertion positions can be considered depending on the requirement, and especially depending on the morphology of the subject or the type of injection required.

It is understood that the insertion position corresponds to the position used to define the product injection depth.

The detection means can provide more than one value relative to the operation of the electric motor. Thus, the detection means can be more precise when monitoring the electric motor by determining several values.

According to other optional characteristics of the insertion device taken alone or in combination:
The insertion device comprises first detection means configured to determine a value relative to the movement of the electric motor, selected from the speed of rotation associated with a duration of rotation, the angle of rotation, the number of revolutions and the number of pulses of the electric motor. Thus, several parameters illustrating the movement of the electric motor, and therefore of the needle support, are possible.
The first detection means comprise an encoder arranged on the shaft of the electric motor and a counter connected to the encoder to count the number of pulses of the electric motor. For a magnetic encoder, a pulse corresponds to one passage of at least one magnet in front of at least one sensor. An optical or audible encoder can also be used.
The insertion device comprises second detection means configured to determine a value relative to the load of the electric motor, selected from the electric current and the torque of the electric motor.

The use of second detection means provides information in addition to that supplied by the first detection means and extends the detection scope in order to detect more phenomena and be more precise regarding the control and management of the needle insertion steps. Note that it would be possible to use only the second detection means, not necessarily combined with the first detection means, bearing in mind that the combination of the two detection means provides more precision. A technical configuration comprising only the second detection means would optimize the volume and overall assembly of the insertion device and therefore the production cost.
The needle support is coupled, at least while the needle is being inserted, with braking means adapted to generate at least one hard point when moving the needle support relative to the body, and in which the electronic means and the second detection means are configured to determine any peak of electric current of the electric motor related to the at least one hard point. More precisely, when the needle support is in the pre-insertion position or in at least one insertion position, a force greater than that necessary to move the needle support is required to cross said pre-insertion position or said at least one insertion position due to the hard point. Each hard point therefore requires additional force to move the needle support.

For example, the peak of electric current can be determined by obtaining a series of electric current values and recording the maximum value measured. When the maximum value decreases, a starting point is marked. If the measured value is equal to the minimum value in the series, it can be considered that the peak of electric current has already been reached.

The braking means can be means for keeping the needle support in its pre-insertion position or means for defining the insertion depth, i.e. the at least one insertion position of the needle support.

The insertion device comprises:
a catheter mounted so as to be movable with respect to the needle and intended to dispense a product into the site,
a pump connected to a reservoir so as to transfer product from the reservoir to the catheter,

the electric motor being configured to supply the pump to transfer the product.

In one embodiment, it is possible to integrate a catheter support adapted to move the catheter with the needle when the needle support changes from its pre-insertion position to the at least one insertion position and to separate it from the needle so that it remains in the at least one insertion position when the needle support changes from the at least one insertion position to the at least one retracted position.

Thus, it is proposed to use very advantageously a single electric motor which performs the insertion function and the product injection function. This reduces the cost and size of the insertion device and facilitates the assembly.
The insertion device comprises a base having a guide housing defining a guide axis, the guide housing being configured to receive the needle support and define at least two hard points, each hard point corresponding to at least one preset depth insertion position. Several insertion, therefore injection, depths are proposed depending on the requirement.

The invention also relates to a method for using an insertion device as described previously, comprising at least one insertion step I, during which the electric motor rotates in a first direction of rotation to move the needle support from its pre-insertion position to the at least one insertion position, and at least the following sub-steps are performed:
detection sub-step D: the detection means detect a first value relative to the movement of the electric motor,
comparison sub-step C: the electronic means compare the first value relative to the movement of the electric motor with a first preset reference to provide information relative to the insertion depth,
in a configuration C-1, if the first value relative to the movement of the electric motor is greater than or equal to the first reference, the electric motor stops rotating, step I is finished;

in a configuration C-2, if the first value relative to the movement of the electric motor is less than the first reference, no modification is made to the direction of rotation of the electric motor.

The first value relative to the movement of the electric motor may be the speed of rotation associated with a duration of rotation, the angle of rotation, the number of revolutions or the number of pulses.

According to other optional characteristics of the use method, taken alone or in combination:

The method comprises a Retraction step R after the configuration C-1, in which the electric motor rotates in a second direction of rotation to move the needle support from the at least one insertion position to the at least one retracted position, and preferably to start the pump, preferably at the same time. It is understood that the second direction of rotation is opposite to the first direction of rotation. Since the needle is retracted and the product dispensed at the same time, the insertion device is even more efficient.
The method comprises a Priming step P before step I, in which the electric motor rotates in a second direction of rotation to start the pump and transfer product from the reservoir to the catheter, and at least the following sub-steps are performed:
detection sub-step Dbis: the first detection means detect the first value relative to the movement of the electric motor,
comparison sub-step Cbis: the electronic means compare the first value relative to the movement of the electric motor with a second preset reference to provide information relative to the priming of the pump,

in a configuration Cbis-1, if the first value relative to the movement of the electric motor is greater than or equal to the second reference, step P is finished and we move to step I;

in a configuration Cbis-2, if the first value relative to the movement of the electric motor is less than the second reference, no modification is made to the direction of rotation of the electric motor.

This priming step can be used to purge the air present in the reservoir or upstream from the catheter, so as to bring product into the catheter. Preferably, in configuration Cbis-1, the electric motor is stopped to change the direction of rotation in order to move to step I.
During the use method, for the insertion step I, the second detection means are used to detect a second value relative to the load of the electric motor, selected from the electric current and the torque of the electric motor, and in which:
first and second peaks of the value relative to the load of the electric motor are detected,
the number of peaks after the first and second peaks is detected, this number of peaks is interpreted as hard point passages and the insertion depth is deduced by the peaks following the first and second peaks.

This is carried out to detect the starting point of the pulse counter. After the priming phase, in fact, when the electric motor changes direction of rotation, the electric current increases, thereby creating a first peak of electric current. The second current peak related to the time when the needle support starts to move occurs after the first peak, when the needle support leaves its “parking” pre-insertion position. Detection of the second peak can make control of the insertion depth more precise.
During step I,
during the detection sub-step D, the second detection means are used to detect a second value relative to the load of the electric motor, selected from the electric current and the torque of the electric motor,
during the sub-step C, the second value relative to the load of the electric motor is compared with a third preset reference, in a configuration C-3, if the second value relative to the load of the electric motor is greater than or equal to the third preset reference, we start to use the first detection means to detect the first value relative to the movement of the electric motor,

in a configuration C-4, if the second value relative to the load of the electric motor is less than the third preset reference, we continue using the second detection means to detect the second value relative to the load of the electric motor.

This makes detection of the insertion depth more precise. It is difficult to precisely determine the relative position of the elements used to move the needle support between them. Depending on this relative position, possible functional play between the complementary drive element and the shaft of the electric motor comprising driving means may affect the first relative value and the accuracy of the interpretation made thereof. The use of a second relative value reduces this inaccuracy.

The peak current is in fact related to the hard point when the needle support leaves the pre-insertion position (“parking” position). The number of pulses necessary to reach the required needle insertion depth is therefore known more precisely. It gives the starting point to count the number of pulses. Without this starting point, there will be an uncertainty due to the functional play between the complementary drive element and the means used to drive the shaft of the electric motor and to the position of the gears.

Thus, this method can be used to check more precisely that the needle support has reached at least one required insertion position.
The method comprises the following steps which follow step R:
   Step Dter: the first detection means detect the value relative to the movement of the electric motor,
   Step Cter: the electronic means compare the first value relative to the movement of the electric motor with a fourth preset reference to provide information relative to the quantity of product dispensed,

in a configuration Cter-1, if the first value relative to the movement of the electric motor is greater than or equal to the fourth reference, the electric motor stops rotating, step R is finished;

in a configuration Cter-2, if the first value relative to the movement of the electric motor is less than the fourth reference, no modification is made to the direction of rotation of the electric motor.

Thus, the needle is retracted and the product dispensed at the same time. Consequently, the activity of the electric motor can be monitored during the various steps of using the insertion device in order to observe and control both displacement of the needle and dispensing of the product. It is also possible to detect any problem related to the pump operation, for example an occlusion in the fluid circuit. In addition, this method provides better control over the quantity of product to be dispensed.

The invention also relates to a kit for assembling a device for inserting a needle into a site comprising:

a body provided with a bearing wall for bearing on the site,

a needle support, the needle support being mounted so as to be movable with respect to the bearing wall between:
a pre-insertion position in which the needle is retracted with respect to the bearing wall,
at least one insertion position in which the needle protrudes with respect to the bearing wall, and
at least one retracted position in which the needle is once again retracted with respect to the bearing wall,
an electric motor comprising a rotation shaft, the electric motor being configured to move the needle support in translation between the different positions of the needle support,
detection means configured to provide a value relative to the operation of the electric motor,
electronic means configured to cooperate with the detection means in order to control the depth of insertion of the needle.

### BRIEF DESCRIPTION OF THE DRAWINGS

It will be easier to understand the invention on reading the description below, given as an example and referring to the attached drawings, on which:

FIG. 1 is a partial perspective view of an insertion device according to one embodiment of the invention;

FIG. 2 is an exploded view of a sub-assembly of the insertion device of FIG. 1;

FIG. 3 is an exploded view of another sub-assembly of the insertion device of FIG. 1;

FIGS. 4a-4e are a set of perspective views showing the various operating steps of the sub-assembly of the insertion device of FIG. 1;

FIGS. 5a-5e are a set of cross-sectional and perspective views showing the operating steps of the sub-assembly of the injection device of FIGS. 4a-4e;

FIG. 6 is a diagram showing an example of the first and second values measured during a complete operating cycle of the insertion device.

### DETAILED DESCRIPTION OF THE INVENTION

FIGS. 1 to 5 show an insertion device according to one embodiment of the invention, designated by the general reference 1. As shown on FIG. 1, the insertion device 1 comprises a body 2 having a bearing wall 19 intended to be placed in direct contact with the skin of a patient. The insertion device 1 is configured to inject a product, preferably liquid, generally a medication, into a site of the patient for a relatively long duration, generally several hours.

The pharmaceutical products likely to be used in the insertion device include for example peptides, proteins, hormones, active substances of biological origin, nucleotide-based active substances, nutritional formulae and other substances.

These active substances may include, but are not limited to, insulins, insulin analogues such as insulin lispro or insulin glargine, insulin derivatives, C-peptide, GLP-1 receptor agonists such as dulaglutide or liraglutide, glucagon, glucagon analogues, glucagon derivatives, gastric inhibitor polypeptides (GIP), GIP analogues, GIP derivatives, oxyntomodulin analogues, oxyntomodulin derivatives, therapeutic antibodies, such as monoclonal antibodies and any therapeutic agent that can be delivered by the above device.

The medication as it is used in the device can be formulated with one or more excipients.

The insertion device 1 is therefore carried by the user during the injection, for example on the waist.

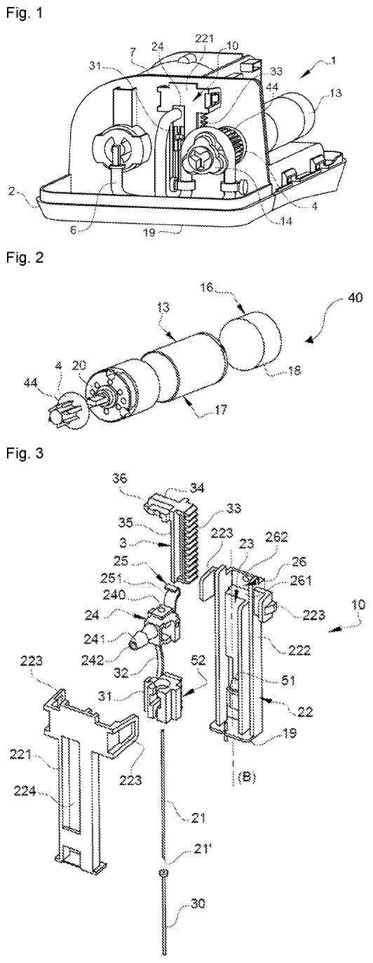

FIG. 2 shows a sub-assembly of the insertion device 1, which is a drive unit 40 comprising, as shown on FIG. 1, an electric motor 13 comprising a shaft 20 fitted with a drive unit 4, 44 which consist, in the example shown, in a rotating element 4 provided with a series of teeth 44 distributed uniformly on the circumference of the rotating element 4. The electric motor 13 can rotate in a first direction of rotation S1 (see FIG. 3c for example) and a second direction of rotation S2 (see FIG. 3d for example). The drive unit 40 further comprises first detection unit 16 comprising, in this example shown, an encoder 18 arranged on the shaft 20 of the electric motor 13 and a counter connected to the encoder 18 to count the number of pulses of the electric motor 13. The insertion device 1 further comprises second detection unit 17 configured to determine a second value relative to the load of the electric motor 13. In the example show, the second detection unit 17 are used to determine the electric current of the electric motor 13.

The insertion device 1 also comprises a pump 14, for example a peristaltic pump as shown on FIG. 1, mounted coaxially with the rotating element 4 and actuated by the same electric motor 13. The pump 14 is connected to a reservoir 7 of product by means of a flexible tube 6 so that when the pump 14 is operated, product is transferred from the reservoir 7 to an injection unit 10 which will be described below. FIG. 1 is a partial view of the insertion device 1 since the latter may comprise a lid or box forming a closed space with the bearing wall 19 so that the elements described previously cannot be seen or accessed from the outside of the insertion device 1.

FIG. 3 shows another sub-assembly of the insertion device 1, which is an injection unit 10. The injection unit 10 comprises a base 22 comprising a first vertical guide 221 and a second vertical guide 222 forming a guide housing 23 defining a guide axis (B). The first vertical guide 221 and the second vertical guide 222 are fixedly mounted to each other, by a clipping member 223 for example. The guide axis (B) can be designed to be substantially perpendicular to the bearing wall 19 or according to any other suitable inclination. In addition, the base 22 comprises, on the first vertical guide 221, a through opening 224 having a longitudinal shape along the guide axis (B). The base 22 also comprises a locking member 51 defining a part of the braking member and carried by the second vertical guide 222 near the bearing wall 19. The functions of the elements carried by the base 22 are described below.

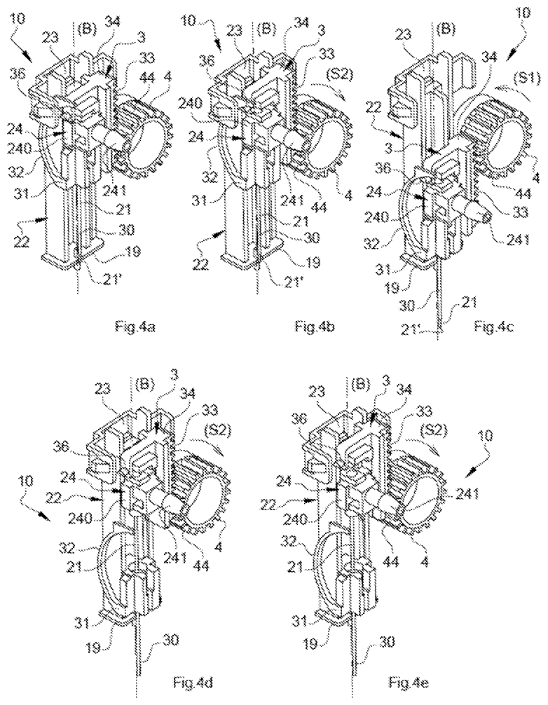

As shown on FIG. 3, the injection unit 10 comprises a needle support 24 mounted so as to be movable in the guide housing 23 between various positions as shown on FIGS. 4 and 5. The needle support 24 comprises a main block 240 of substantially cross-shaped section along a plane parallel to the bearing wall 19. In addition, the shape of the main block 240 is substantially complementary to that of the guide housing 23, so that the needle support 24 can slide in the guide housing 23 along the guide axis (B).

The needle support 24 is coupled, at least while the needle is being inserted, with braking member adapted to generate hard points when moving the needle support 24 relative to the body 2.

For example, the needle support 24 comprises a blocking member 25, defining another part of the braking member, consisting of an elastic hook 251 mounted on a flexible portion, the blocking member 25 being configured to cooperate with complementary blocking member 26 also defining a part of the braking member and comprising a first vertical stop 261 and a second vertical stop 262 carried by the base 22, the cooperation between the blocking member 25 and the complementary blocking member 26 being shown more clearly on FIG. 5. When the needle support 24 is moved so that the elastic hook 251 is no longer engaged by the first vertical stop 261 or the second vertical stop 262, a hard point is generated and generates a peak of electric current which can be detected by the second detection unit 17.

As shown on FIGS. 3 to 5, the needle support 24 also comprises a protuberance 241 intended to cross the longitudinal through opening 224 and to slide therein. The protuberance 241 is intended to be connected to the tube 6 as shown on FIG. 1 to allow fluid communication with the reservoir 7 and the product it contains. To do this, the protuberance 241 is provided with a dispensing channel 242 as shown on FIG. 5 to transfer product into the needle support 24. An insertion needle 21 is fixedly mounted to the needle support 24 and is provided with a lateral orifice in fluid communication with the dispensing channel 242, thus allowing the product to be injected to reach the insertion end 21′ and therefore the injection site once the needle 21 has been inserted.

According to the invention, the needle support 24 is mounted so as to be movable with respect to the bearing wall 19 between:
a pre-insertion position in which the needle 21 is retracted with respect to the bearing wall 19,
at least one insertion position in which the needle 21 protrudes with respect to the bearing wall 19, and
at least one retracted position in which the needle 21 is once again retracted with respect to the bearing wall 19.

To do this, the injection unit 10 comprises a complementary drive element 3 (see FIG. 3 for example) intended to translate the needle support 24 between the various positions of the needle support 24. As shown on FIGS. 3 to 5, the complementary drive element 3 is L-shaped and comprises a first horizontal portion 34 which extends along an axis perpendicular to the guide axis (B), and a second vertical portion 35 which extends along an axis parallel to the guide axis (B). The first portion 34 has a flat surface as shown on FIG. 5 intended to come into contact with the needle support 24 and to move the needle support 24. The first portion 34 further comprises a locking pin 36 (see FIG. 4a for example) arranged at one end of the first portion 34 opposite the second portion 35. The second portion 35 is provided with a rack 33 (see FIG. 3 for example) intended to cooperate with the series of teeth 44 on the rotating element 4 so that the complementary drive element 3 can be driven in translation along the guide axis (B) in the two directions opposite to each other and according to the first and second directions of rotation S1, S2 of the electric motor 13.

The injection unit 10 also comprises a catheter 30 carried by a catheter support 31, as shown on FIGS. 3 to 5. In the example shown, in particular on FIGS. 3 and 4, the catheter support 31 comprises an arcuate-shaped elastic element 32, having one end attached to the catheter support 31 and the other end forming a stop intended to cooperate with the locking pin 36 so that the rack 33 continues to interact with the series of teeth 44. Advantageously, the elastic element 32 and the locking pin 36 no longer interact when the rack 33 and the needle support 24 are retracted after insertion of the needle 21 and rotation of the electric motor 13 according to a second direction of rotation S2 (see FIGS. 3d and 3e).

As shown on FIG. 5, the catheter support 31 comprises notches 52 configured to cooperate with the locking member 51 to keep the catheter support 31 in a chosen insertion position. When a notch crosses the locking member 51, a hard point is generated and generates a peak of electric current which can be detected by the second detection unit 17. Thus, the locking member 51 forms the braking member.

The catheter support 31 is connected in translation to the needle support 24 during insertion of the needle 21. Since the needle 21 is housed inside the catheter 30, insertion of the needle 21 into the site allows simultaneous insertion of the catheter 30.

FIGS. 4 and 5 show the various operating steps of the insertion device 1, which will be described as follows: when the injection device 1 is ready to be used by a patient or by a member of the medical staff, the needle support 24 is in a pre-insertion position in which the needle 21 is retracted with respect to the bearing wall 19. The needle support 21 is kept in this pre-insertion position by the interaction between the blocking member 25, in this case consisting of an elastic hook 251 and the complementary blocking member 26 (FIGS. 3a and 4a). The catheter support 31 and the complementary drive element 3 are also kept in this pre-insertion position since they are respectively against the needle support 21 on one side and blocked in translation by the interaction between the elastic element 32 and the locking pin 36 carried by the complementary drive element 3 on the other side.

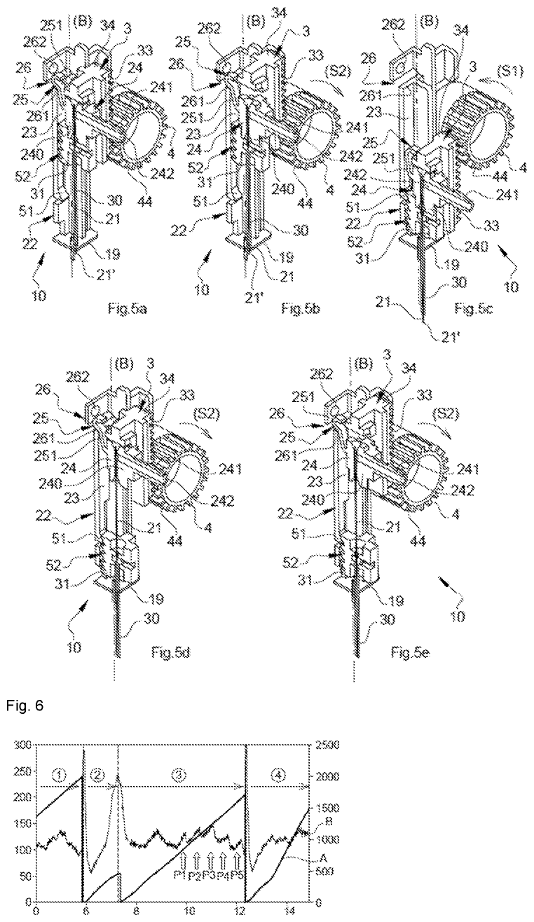

FIG. 6 shows the first (line A) and second (line B) values measured during these steps. The horizontal axis (abscissae) corresponds to a time line in seconds, the vertical axis (ordinates) on the left corresponds to the quantities of the electric current in milliamps and the vertical axis on the right corresponds to the number of pulses.

When the injection device 1 is ready to be used by a patient or by a member of the medical staff, the needle support 24 is in a pre-insertion position in which the needle 21 is retracted with respect to the bearing wall 19. The needle support 21 is kept in this pre-insertion position by the interaction between the hook 251 and the complementary blocking member 26 (FIGS. 4a and 5a). The catheter support 31 and the complementary drive element 3 are also kept in this pre-insertion position since they are respectively against the needle support 21 on one side and blocked in translation by the interaction between the elastic element 32 and the locking pin 36 carried by the complementary drive element 3 on the other side.

When the user places the insertion device 1 on a site to be injected, the bearing wall 19 is for example in direct contact with the site. The user then activates the electric motor 13, using a control button for example, to rotate the electric motor 13 as well as the rotating element 4 in the second direction of rotation S2 as shown on FIGS. 4b and 5b. The insertion device 1 is then in priming phase, during which the pump 14 starts to transfer product from the reservoir 7 towards the catheter 30, successively via the tube 6, the pump 13 and the needle 21. This priming phase is programmed to last for a short period of time, but sufficient to purge the air present in the reservoir 7 or upstream from the catheter 30.

During the priming phase, the series of teeth 44 carried by the rotating element 4 in contact with the rack 33 carried by the complementary drive element 3 drives the latter in an upward direction with respect to the guide axis (B), i.e. in a direction away from the bearing wall 19. Simultaneously, the elastic element 32 presses down on the locking pin 36, i.e. in a downward direction with respect to the guide axis (B) and therefore towards the bearing wall 19. Thus, the complementary drive element 3 exerts a very small back and forth movement so that the complementary drive element 3 continues to interact with the series of teeth 44 of the rotating element 4. The complementary drive element 3 is therefore brought into contact with the drive exerted by the rotation of the rotating element 4 in the second direction of rotation S2 and can therefore continue priming the insertion device 1. This priming phase is shown on the diagram of FIG. 6 and is indicated by the digit “1”. During the priming phase, the number of pulses measured increases and the electric current remains stable. The insertion device 1 comprises an electronic unit 8 configured to cooperate with the first detection unit 16. Thus, when the number of pulses measured by the counter is greater than or equal to a second preset reference which is a number of reference pulses relative to the priming, the electronic member 8 controls the electric motor 13 to stop the priming and move to an insertion step. Otherwise, the electric motor 13 continues to rotate in the second direction of rotation S2.

When priming of the insertion device 1 is finished, the electric motor 13 rotates in a first direction of rotation 51, which is opposite to the second direction of rotation S2, to drive the needle support 24 towards a preset insertion position. The change in the direction of rotation of the electric motor 13 increases the electric current and generates a first peak of electric current which is shown on FIG. 6, at the end of phase “1”.

Then, the series of teeth 44 engages with the rack 33 and converts the rotation of the rotating element 4 into translation of the complementary drive element 3 towards the bearing wall 19. The complementary drive element 3 itself drives the needle support 24 and the catheter support 31 in the same direction towards the bearing wall 19 as shown on FIGS. 4c and 5c. When the needle support 24 leaves its pre-insertion position and moves towards the bearing wall 19, the blocking member 25 no longer interact with the complementary blocking member 26, since the blocking member 25 comprises an elastic hook 251 which is held completely inside the guide housing 23 as shown on FIG. 5c. To separate the blocking member 25 from the complementary blocking member 26, a greater force must be supplied by the electric motor 13, which increases the electric current and generates a second peak of electric current which is shown on FIG. 6, at the end of phase

Detection of the second peak of electric current resets the pulse counter as shown on FIG. 6 and starts the first detection unit 16 to measure the number of pulses from this precise time, as shown by curve A on FIG. 6.

When moving the needle support 24 to one of the insertion positions, the electronic unit 8 compares the number of pulses measured by the counter with a first preset reference which is a number of reference pulses relative to the insertion.

When the needle support 24 has nearly reached the bearing wall 19, the notches 52 carried by the catheter support 31 start to come into contact with the locking member 51, one after the other. If the needle support 24 reaches the preset insertion position, the corresponding notch which is already in contact with the locking member 51 continues to cooperate with the locking member 51 to keep the catheter support 31 in position. In this configuration, the number measured is greater than or equal to this number of reference pulses relative to the insertion, the electric motor 13 stops rotating and insertion step I is finished. If the needle support 24 has not reached the preset insertion position, the electric motor 13 continues to rotate in the first direction of rotation S1 and the rack 33 continues to move until the needle support 24 reaches the preset insertion position. FIGS. 5c to 5e show an insertion position in which the last notch which comes into contact with the locking member 51 cooperates with the latter to keep the catheter support 31 in a position which corresponds to a “shallow” insertion position of the needle 21, equivalent for example to the subcutaneous layer of the patient's skin. According to the example shown, the needle support 24 can have up to 5 different insertion positions. Consequently, 5 hard points related to the insertion of the needle can be generated. The current peaks generated by the hard points related to the insertion of the needle are shown on FIG. 6, indicated respectively by the arrows P1, P2, P3, P4 and P5 and the insertion phase is indicated by the digit “3” on the same figure.

Once the catheter 30 has been inserted into the site by inserting the needle 21, the needle support 24 separates from the catheter support 31 and moves from its insertion position to its retracted position. To do this, the electric motor 13 rotates again in the second direction of rotation S2 so that the drive unit 4, 44 drives the complementary drive element 3 and the needle support 24 in an upward direction with respect to the guide axis (B), towards the retracted position of the needle support 24. Since the driving force of the electric motor 13 is greater than the clipping between the notches 52 and the locking member 51, the elastic element 32 and the locking pin 36 no longer cooperate and the complementary drive element 3 separates from the catheter support 31. The change in the direction of rotation of the electric motor 13 increases the electric current and generates a third peak of electric current which is shown on FIG. 6, at the end of phase “3”. Detection of the third peak of electric current resets the pulse counter again as shown on FIG. 6 and starts the first detection unit 16 to measure the number of pulses from this precise time.

The needle support 24 is connected in translation with the complementary drive element 3 by the hooking formed by the blocking member 25 via the elastic hook 251 on the first horizontal portion 34 of the complementary drive element 3.

As the needle 21 is being removed, the electric motor 13 rotates in the second direction of rotation S2 and therefore operates the pump 14 in order to transfer a preset quantity of product from the reservoir 7 to the catheter 30. This corresponds to phase “4” as shown on FIG. 6. The number of pulses measured by the counter increases and the electric current remains stable. The electronic unit 8 compares the number of pulses measured by the counter with a third preset reference which is a number of reference pulses relative to the dispensing of the product. When the number of pulses measured by the counter is greater than or equal to the third preset reference, the electronic unit 8 stops the electric motor 13. Otherwise, the electric motor 13 continues to rotate in the second direction of rotation S2.

When the needle support 24 reaches the retracted position, the elastic hook 251 and the complementary blocking member 26 cooperates again to lock the needle support 24 in this retracted position, as shown on FIGS. 4d and 5d. Since the elastic hook 251 is no longer in contact with the first horizontal portion 34 of the complementary drive element 3, the latter becomes movable with respect to the needle support 24. The needle support 24 is then stable in the retracted position where it is held. Thus, the electric motor 13 can continue to rotate in the second direction of rotation S2 in order to finalize the transfer of the preset quantity of product.

The injection device 1 is for example assembled using an assembly kit comprising:
a body 2 provided with a bearing wall 19 for bearing on the site,
a needle support 24, the needle support 24 being mounted so as to be movable with respect to the bearing wall 19 between:
   a pre-insertion position in which the needle 21 is retracted with respect to the bearing wall 19,
at least one insertion position in which the needle 21 protrudes with respect to the bearing wall 19, and
at least one retracted position in which the needle 21 is once again retracted with respect to the bearing wall 19,
an electric motor 13 comprising a rotation shaft 20, the electric motor 13 being configured to move the needle support 24 in translation between the different positions of the needle support 24,
a detection unit 16, 17 configured to provide a value relative to the operation of the electric motor 13,
an electronic unit 8 configured to cooperate with the detection unit 16, 17 in order to control the depth of insertion of the needle 21.

The invention is not limited to the embodiments described and other embodiments will be clearly apparent to those skilled in the art.

1. A insertion device for inserting a needle into a site comprising:
a body provided with a bearing wall for bearing on the site,
a needle support on which a needle is mounted, the needle support being mounted so as to be able to move relative to the bearing wall between:
a pre-insertion position in which the needle is retracted with respect to the bearing wall,
at least one insertion position in which the needle protrudes with respect to the bearing wall, and
at least one retracted position in which the needle is once again retracted with respect to the bearing wall,
an electric motor comprising a rotation shaft, the electric motor being configured to move the needle support in translation between the different positions of the needle support,
at least one detection unit configured to provide a value relative to the operation of the electric motor,
an electronic unit configured to cooperate with the at least one detection unit to control a depth of insertion of the needle.

2. The insertion device according to claim 1, wherein the at least one detection unit comprises a first detection unit configured to determine a value relative to the movement of the electric motor, selected from a speed of rotation associated with a duration of rotation, an angle of rotation, a number of revolutions and a number of pulses of the electric motor.

3. The insertion device according to the claim 2, wherein the first detection unit comprises an encoder arranged on the shaft of the electric motor and a counter connected to the encoder to count the number of pulses of the electric motor.

4. The insertion device according to claim 2, wherein the at least one detection unit further comprises a second detection unit configured to determine a value relative to a load of the electric motor, selected from an electric current and a torque of the electric motor.

5. The insertion device according to claim 4, wherein the needle support is coupled, at least while the needle is being inserted, with a braking member adapted to generate at least one hard point when moving the needle support relative to the body, and wherein the electronic unit and the second detection unit are configured to determine any peak of electric current of the electric motor related to the at least one hard point.

6. The insertion device according to claim 1, comprising:
a catheter mounted so as to be movable with respect to the needle and configured to dispense a product into the site,
a pump connected to a reservoir so as to transfer product from the reservoir to the catheter,
the electric motor being configured to supply the pump to transfer the product.

7. The insertion device according to claim 5, further comprising a base having a guide housing defining a guide axis, the guide housing being configured to receive the needle support and define at least two hard points, each hard point corresponding to at least one preset depth insertion position.

8. A method for using the insertion device according to claim 1, comprising at least one insertion insertion step, during which the electric motor rotates in a first direction of rotation move the needle support from the pre-insertion position to the at least one insertion position, and at least the following sub-steps are performed:
a detection sub-step D, during which the detection unit detects a first value relative to the movement of the electric motor, and
a comparison sub-step C, during which the electronic unit compares the first value relative to the movement of the electric motor with a first preset reference to provide information relative to the insertion depth,
wherein in a configuration C-1, if the first value relative to the movement of the electric motor is greater than or equal to the first preset reference, the electric motor stops rotating, and the at least one insertion step is finished;
and
wherein in a configuration C-2, if the first value relative to the movement of the electric motor is less than the first preset reference, no modification is made to the direction of rotation of the electric motor.

9. The method for using the insertion device, according to 8, wherein the insertion device further comprises a catheter mounted so as to be movable with respect to the needle and configured to dispense a product into the site, and a pump connected to a reservoir so as to transfer product from the reservoir to the catheter, the electric motor being configured to supply the pump to transfer the product, wherein the method further comprises a retraction step after the configuration C-1, wherein the electric motor rotates in a second direction of rotation to move the needle support from the at least one insertion position to the at least one retracted position and to start the pump at the same time.

10. The method for using the insertion device according to claim 9, wherein the at least one detection unit comprises a first detection unit configured to determine a value relative to the movement of the electric motor, selected from a speed of rotation associated with a duration of rotation, an angle of rotation, a number of revolutions and a number of pulses of the electric motor, the method further comprising a priming step before the at least one insertion step, wherein the electric motor rotates in a second direction of rotation to start the pump and to transfer product from the reservoir to the catheter, and at least the following sub-steps are performed:
a detection sub-step Dbis, during which the first detection unit detects the first value relative to the movement of the electric motor, and
a comparison sub-step Cbis, during which the electronic unit compares the first value relative to the movement of the electric motor with a second preset reference to provide information relative to the priming of the pump,
wherein in a configuration Cbis-1, if the first value relative to the movement of the electric motor is greater than or equal to the second preset reference, the priming step is finished and to the insertion step is performed; and
wherein in a configuration Cbis-2, if the first value relative to the movement of the electric motor is less than the second preset reference, no modification is made to the direction of rotation of the electric motor.

11. The method for using the insertion device according to claim 8, wherein the at least one detection unit further comprises a second detection unit, wherein the needle support is coupled, at least while the needle is being inserted, with a braking member adapted to generate at least one hard point when moving the needle support relative to the body, wherein the electronic unit and the second detection unit are configured to determine any peak of electric current of the electric motor related to the at least one hard point, and wherein during the insertion step, the second detection unit is used to detect a second value relative to a load of the electric motor, selected from an electric current and a torque of the electric motor, and wherein:
first and second peaks of the second value relative to the load of the electric motor are detected, and
a number of peaks after the first and second peaks is detected, and this number of peaks is interpreted as hard point passages and an insertion depth is deduced by the peaks following the first and second peaks.

12. The method for using an insertion device according to claim 8, wherein the at least one detection unit further comprises a second detection unit, wherein
during the detection sub-step D, the second detection unit is used to detect a second value relative to a load of the electric motor, selected from an electric current and a torque of the electric motor,
during the sub-step C, the second value relative to the load of the electric motor is compared with a third preset reference,
in a configuration C-3, if the second value relative to the load of the electric motor is greater than or equal to the third preset reference, the first detection unit is used to detect the first value relative to the movement of the electric motor;
in a configuration C-4, if the second value relative to the load of the electric motor is less than the third preset reference, the second detection unit is continued to be used to detect the second value relative to the load of the electric motor.

13. The method for using an insertion device according to claim 9, comprising the following steps which follow the retraction step:
step Dter, during which the first detection unit detects the first value relative to the movement of the electric motor,
step Cter, during which; the electronic unit compares the first value relative to the movement of the electric motor with a fourth preset reference to provide information relative to aquantity of product dispensed, wherein in a configuration Cter-1, if the first value relative to the movement of the electric motor is greater than or equal to the fourth preset reference, the electric motor stops rotating and the insertion step is finished; and
wherein in a configuration Cter-2, if the first value relative to the movement of the electric motor is less than the fourth preset reference, no modification is made to the direction of rotation of the electric motor.

14. A kit for assembling an insertion device for inserting a needle into a site, comprising:
a body provided with a bearing wall for bearing on the site,
a needle support, the needle support being mounted so as to be movable with respect to the bearing wall between:
a pre-insertion position in which the needle is retracted with respect to the bearing wall,
at least one insertion position in which the needle protrudes with respect to the bearing wall, and
at least one retracted position in which the needle is once again retracted with respect to the bearing wall,
an electric motor comprising a rotation shaft, the electric motor being configured to move the needle support in translation between the different positions of the needle support,
a detection unit configured to provide a value relative to the operation of the electric motor, and
an electronic unit configured to cooperate with the detection unit to control a depth of insertion of the needle.
